(19) United States
(12) Patent Application Publication
(10) Pub. No.: US 2009/0232755 A1
(43) Pub. Date: Sep. 17, 2009

(54) COMBINATION THERAPIES FOR TREATING PHOTODAMAGED SKIN

(76) Inventor: Leslie Baumann, Miami Beach, FL (US)

Correspondence Address: LOUIS C. PAUL, 420 East 61st Street, 8E, NEW YORK, NY, 10021, US

(21) Appl. No.: 12/403,249

(22) Filed: Mar. 12, 2009

Related U.S. Application Data
(63) Continuation-in-part of application No. 10/627,994, filed on Jul. 28, 2003, now Pat. No. 7,521,459.

(51) Int. Cl. A61K 8/00; A61K 31/4745; A61Q 17/04

(52) U.S. Cl. 424/59; 514/293

(57) ABSTRACT

Combination therapies for reducing the appearance of fine lines and wrinkles on aged skin or non-precancerous, normal photodamaged section of skin, in a patient not being treated for viral infection or skin cancer comprising (i) topical application of an imidazoquinoline amine derivative in a dermatologically-acceptable carrier in further combination with one or more cosmetic treatments selected from the group consisting of: (i) Light Emitting Diode (L.E.D.) Light Therapy; (ii) Intense Pulsed Light (I.P.L.) Therapy; (iii) laser skin resurfacing; (iv) mechanical exfoliation; (v) superficial, medium depth or deep chemical peels; (vi) radiofrequency treatment; (vii) ultrasound treatment; (viii) intradermal and intraepidermal injections with hyaluronic acid and derivatives thereof; and (ix) cryosurgery.

### CROSS-REFERENCE TO RELATED APPLICATIONS

This is a continuation in part of U.S. application Ser. No. 10/627,994 the disclosure of which is incorporated herein by reference in its entirety.

### STATEMENT REGARDING FEDERALLY-SPONSORED RESEARCH OR DEVELOPMENT

Not applicable.

### FIELD OF THE INVENTION

The present invention relates to methods and compositions for treating intrinsically and extrinsically-aged skin, including, in particular, facial skin having fine lines or wrinkles, as well as normal photodamaged skin

### BACKGROUND OF THE INVENTION

Topically-applied imiquimod (5%) has been approved by the FDA for the treatment of three dermatologic conditions: (i) clinically typical, nonhyperkeratotic, nonhypertrophic actinic keratoses (“AKs”) on the face or scalp in immunocompetent adults; (ii) biopsy-confirmed, primary superficial basal cell carcinoma in immunocompetent adults, with a maximum tumor diameter of 2.0 cm, located on the trunk (excluding anogenital skin), neck, or extremities (excluding hands and feet), when surgical methods are medically less appropriate and patient follow-up can be reasonably assured; and (iii) external genital and perianal warts in patients 12 years and older. The first two indications were approved in March 2004 and July 2004, respectively.

Actinic keratoses are “epidermal tumours which result from the proliferation of transformed neoplastic keratinocytes.” See Stockfleth et al., “Successful treatment of actinic keratosis with imiquimod cream 5%: a report of six cases,” Br. J. Dermatol., Vol. 144, pp. 1050-1053 (2001). AKs can progress into thickened or hypertrophic lesions, which can subsequently develop into squamous cell carcinomas. AKs are, therefore, sometimes referred to as “precancerous lesions.” Clinically, AKs present as easily palpable, rough or “gritty,” usually erythematous patches, ranging in size from several millimeters up to about one centimeter in diameter. Histologically, AKs are basophilic and variable in shape and size, with large, polymorphic and heterochromatic nuclei. (The latter is sometimes referred to as “nuclear atypia.”) AKs are also characterized by dysplasia and loss of cellular polarity.

Normal (i.e., non-precancerous) photodamaged skin and aged skin differ from AKs, both in clinical and histological presentation. Aged skin results from both intrinsic and extrinsic factors. Intrinsic aging is manifested as fine lines and deepening of facial expression lines. Intrinsically-aged skin is thin and inelastic. Extrinsic aging can cause aged skin to appear yellowed and blemished and have mottled pigmentation, coarse wrinkles and furrowing. On physical examination, extrinsically-aged skin is thickened, lax, rough and leathery. Histologically, aged skin shows varying degrees of cytological atypia (both of keratinocytes and melanocytes). On microscopic examination, normal, photodamaged skin exhibits elastosis—thickened, twisted degraded elastic fibers which, over time, degenerate into an amorphous mass. The histology of normal photodamaged skin is also characterized by a decrease in the quantity of collagen fibers and the presence of inflammatory infiltrate. Intrinsically-aged skin is characterized histologically by a flattening of rete pegs at the dermoepidermal junction. Z D Draelos, “Topical Treatments for Benign Photodamage” in D J Goldberg (ed.), Photodamaged Skin, pp. 146-147 (2004).

Many cytokines and growth factors such as interferon, tumor necrosis factor, interleukin 1, and interleukin 12 are induced by imiquimod. See, e.g., L M Imbertson et al., “Cytokine induction in hairless mouse and rat skin after topical application of immune response modifiers imiquimod and S-28463,” J. Invest. Dermatol. Vol. 110, pp. 734-739 (1998); see also, U R Hengge et al., “Topical immunomodulators-progress towards treating inflammation, infection, and cancer,” Lancet Infect. Dis. Vol. 1, pp. 189-198 (2001).

A 2005 review article by Vender entitled “Innovative Uses of Imiquimod” reports on the successful treatment of over forty dermatologic conditions, anecdotally or in clinical trial settings. Journal of Drugs in Dermatology, Vol. 4, No. 1, pp. 58-63. Treatment of normal photodamaged skin (i.e., non-precancerous skin) is not mentioned. Nor is treatment of fine lines and wrinkles in aged skin.

In March 2006, Drs. Albert Kligman and Raymond Cornelison, Jr. presented a poster paper relating to the cosmetic use of imiquimod at the 64th Annual Meeting of the American Academy of Dermatology in San Francisco. The study is described in an article entitled “Topical Imiquimod Improves Cosmetic Appearance of Photoaged Skin” published in the Apr. 1, 2006 edition of Dermatology Times: “To Dr. Kligman's surprise, the treatment caused no local adverse reactions. However, it did result in improvements in the appearance of fine lines and wrinkles, skin texture and dyschromia that were consistent with histologic studies showing correction of epidermal dysplasia.”

In a 2007 study relating to the use of imiquimod in the treatment of lentigo maligna, Metcalf et al. reported that topical imiquimod appears to induce reparative changes to the epidermis and the dermal collagen table in chronically sun-damaged skin associated with lentigo maligna, indicating the potential use of imiquimod as an antiaging treatment. “Imiquimod as an antiaging agent,” J. Amer. Acad. Derm. Vol. 56, No. 3, pp. 422-425 (March 2007).

In the background section of an article investigating histologic and immunohistologic changes in actinically-damaged skin after treatment with imiquimod, Smith et al. noted that imiquimod (5%) is believed by some to result in an improved cosmetic appearance of chronically UV radiation damaged skin. “Does Imiquimod Histologically Rejuvenate Ultraviolet Radiation-Damaged Skin,” Dermatologic Surgery, Vol. 33, No. 12, pg. 1419 (December 2007).

The use of hydroxy-acids (alpha, beta and polyhydroxy) to treat photodamaged skin is well-known in dermatology. See, e.g., R J Yu and E J Van Scott, “Chapter 9: α-Hydroxyacids, Polyhydroxyacids, Aldobionic Acids and their Topical Actions,” in R. Baran and H J Maibach (eds.), Textbook of Cosmetic Dermatology, pp. 77-93 (2004). See also, C M Dietre, “Effects of alpha-hydroxy acids on photoaged skin,” J. Am. Acad. Dermatol., Vol. 34, pp. 187-195 (1996). Classified as monocarboxylic (glycolic, lactic, mandelic), dicarboxylic (malic and tartaric), and tricarboxylic (citric), alpha hydroxy acids (AHAs) cause corneocyte disadhesion, specifically in the stratum corneum. See, e.g., E. Berardesca, “AHA mechanism of action,” Cosmetics & Toiletries, Vol. 110, pp. 30-31 (1995). Salicylic acid, a beta hydroxy acid (BHA), can induce exfoliation. It is a comedolytic approved by the FDA for the treatment of acne.

Topical retinoids are effective in treating clinical signs of photoaging, including fine wrinkles, coarseness and skin laxity. See, e.g., L. Rittié, G J Fisher and J J Voorhees, “Chapter 13: Retinoid Therapy for Photoaging” in B A Gilchrest and J Kruttman (eds) Skin Aging (2006).

### SUMMARY OF THE INVENTION

The present invention relates to the combination therapy for treating fine lines or clinical wrinkles on a section of aged skin or non-precancerous, normal photodamaged skin, comprising
(a) topically applying, in a dermatologically-acceptable carrier, a safe and effective amount of an imidazoquinoline amine derivative conforming to the structure

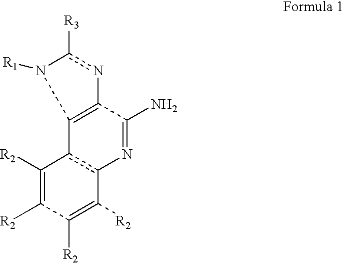

wherein
(i) R₁ is selected from the group consisting of C₁-C₁₀ alkyl; C₁-C₆ hydroxylalkyl; and acyloxyalkyl wherein the acyloxy moiety is C₂-C₄ alkanoyloxy or benzoyloxy, and the alkyl moiety contains one to six carbon atoms or a benzyl, (phenyl)ethyl or phenyl
(ii) R₂ is hydrogen or no more than two non-hydrogen moieties selected from the group consisting of C₁-C₄ alkyl, C₁-C₄ alkoxy, and halogen with the proviso that non-hydrogen moieties are present then said moieties together contain no more than 6 carbon atoms;
(iii) R₃ is selected from the group consisting of hydrogen, C₁-C₈ alkyl, benzyl, (phenyl)ethyl and phenyl; and
(b) topically applying, in a dermatologically-acceptable carrier, a safe and effective amount of a retinoid, a hydroxyacid and combinations thereof, optionally in further combination with a growth factor and/or an agent that attenuates or blocks ultraviolet radiation from 280-400 nm and/or
(c) administering to fine lines or clinical wrinkles on a section of aged skin or non-precancerous, normal photodamaged skin one or more cosmetic treatments selected from the group consisting of:
   (i) Light Emitting Diode (L.E.D.) Light Therapy
   (ii) Intense Pulsed Light (I.P.L.) Therapy
   (iii) laser skin treatments;
   (iv) mechanical exfoliation;
   (v) superficial, medium depth or deep chemical peels, including with chemicals selected from the group consisting of glycolic acid, salicylic acid, trichloroacetic acid, phenol and Jessner's solution; and
   (vi) radiofrequency treatment;
   (vii) ultrasound treatment;
   (viii) intradermal and intraepidermal injections (e.g., mesotherapy with hyaluronic acid and derivatives thereof); and
   (ix) cryosurgery.

### DETAILED DESCRIPTION OF THE INVENTION

A first aspect of the present invention relates to the combination therapy of treating fine lines or clinical wrinkles on a section of aged skin or non-precancerous, normal photodamaged skin by topical application of safe and effective amounts of imiquimod (or another imidazoquinoline amine derivative according to Formula 1) in combination with one or more retinoids, hydroxyacids, growth factors and/or sunscreens or sunblocks.

As used in the present application, “aged skin” means “intrinsically-aged skin” and “extrinsically-aged skin.” “Intrinsically-aged skin” means skin that is thin and inelastic. “Extrinsically-aged skin” means skin that is thickened, lax, rough and leathery. Extrinsically-aged skin aging may appear yellowed and blemished and/or have mottled pigmentation, coarse wrinkles and furrowing. Histologically, aged skin shows varying degrees of cytological atypia (both of keratinocytes and melanocytes).

As used in the present application, the “normal, photodamaged skin” means skin that after exposure to ultraviolet radiation in the spectrum 280-400 nm exhibits elastosis—thickened, twisted degraded elastic fibers which, over time, degenerate into an amorphous mass. Histologically, “normal, photodamaged skin” is characterized by a decrease in the quantity of collagen fibers and the presence of inflammatory infiltrate.

As used herein, “safe and effective amount” means (i) a sufficient amount of a compound or composition to induce a clinically positive modification in the condition being treated (here photodamage), as determined based on the professional judgment of a person having ordinary skill in the art (e.g., a dermatologist), that is (ii) low enough to avoid significant side effects (e.g., significant skin irritation or sensitization). The safe and effective amount of the compound or composition may vary with the particular skin type being treated, the age and physical condition of the patient being treated; the severity of the condition, the duration of the treatment, the nature of concurrent therapy, the specific compound or composition being administered, the particular dermatologically-acceptable carrier utilized, and similar factors within the knowledge and expertise of the skilled artisan.

The term “dermatologically-acceptable,” as used in the present application, means that the compositions and ingredients thereof are suitable for use in contact with mammalian skin without irritation, sensitization, toxicity, incompatibility, instability, allergic response, or other adverse effects.

A preferred imidazoquinoline amine derivative according to Formula 1 suitable for use in the methods of combination therapy of the present invention is 1-isobutyl-1H-imidazo[4,5-c]quinolin-4-amine, also known imiquimod. Imiquimod may be administered at concentrations of from about 0.1% to about 5.0% on a daily basis, as well as one or more times a week but less than once a day (e.g., once every other day, or once every third day, or twice a week application).

Retinoids are well known in the art and are commercially available from a number of sources, including Sigma Chemical Company (St. Louis, Mo.). As used in the present application, the term “retinoid” is meant to include all natural and/or synthetic analogs of Vitamin A or retinol-like compounds which possess the biological activity of Vitamin A in the skin as well as the geometric isomers and stereoisomers of these compounds, such as all-trans retinoic acid and 13-cis-retinoic acid. Retinoids suitable for use in the present invention may be selected from the group consisting of retinol, retinal, retinol esters (C₂-C₂₂ alkyl esters of retinol, including retinyl palmitate, retinyl acetate, retinyl propionate), retinal, and/or retinoic acid (including all-trans retinoic acid and/or 13-cis-retinoic acid).

Retinoids in addition to those listed in the immediately paragraph which may be used in the methods and compositions of the present invention include tocopheryl-retinoate [tocopherol ester of retinoic acid (trans- or cis-), adapalene {6-[3-(1-adamantyl)-4-methoxyphenyl]-2-naphthoic acid}, tazarotene (ethyl 6-[2-(4,4-dimethylthiochroman-6-yl)-ethynyl]nicotinate as well as the retinoids described in the following U.S. patents, the disclosures of which are incorporated herein by reference in their entirety: U.S. Pat. No. 4,677,120; U.S. Pat. No. 4,885,311; U.S. Pat. No. 5,049,584; U.S. Pat. No. 5,124,356.

One or more retinoids may be used in the combination therapies of the present invention. Preferred retinoids are retinol, retinyl palmitate, retinyl acetate, retinyl propionate, retinal and combinations thereof.

In the combination therapies of the present invention, retinoids may be used at concentrations of from about 0.001% to about 10%, by weight of the topical composition in which they are formulated. Preferably retinoids are administered at a concentration of from about 0.001% to about 5% by weight of the composition, more preferably from about 0.01% to about 5%, most preferably from about 0.01% to about 3%. (All percentages and ratios used herein, unless otherwise indicated, are by weight.)

AHAs, hydroxy acids in which the hydroxy group is attached to the alpha carbon atom of the acid, conform to the structure: (R₁)(R₂)C(OH)COOH, where R₁ and R₂ are selected from the group consisting of hydrogen, alkyl, aralkyl and aryl groups, the latter groups (alkyl, aralkyl and aryl) having from 1 to 29 carbon atoms. The alkyl, aralkyl and aryl groups may be saturated or unsaturated, isomeric or non-isomeric, straight or branched chain or cyclic. These groups may also contain as substituents OH, CHO, COOH and alkoxy groups having from 1 to 9 carbon atoms. R₁ and R₂ may be the same or different. In the latter case, the AHAs may be stereoisomers in the D, L, and DL forms. R₁ and R₂ may also be Cl, Br, I, S, F, or an alkyl or alkoxy group, saturated or unsaturated, having 1 to 9 carbon atoms. As used in the present application, the term “AHA” means not only the free acid, but also its corresponding esters, lactones and salts.

AHAs suitable for use in the present invention include: 2-hydroxyethanoic acid (glycolic acid, hydroxyacetic acid); 2-hydroxypropanoic acid (lactic acid); 2-methyl 2-hydroxypropanoic acid (methyllactic acid); 2-hydroxybutanoic acid; 2-hydroxypentanoic acid; 2-hydroxyhexanoic acid; 2-hydroxyheptanoic acid; 2-hydroxyoctanoic acid; 2-hydroxynonanoic acid; 2-hydroxydecanoic acid; 2-hydroxyundecanoic acid; 2-hydroxydodecanoic acid (alpha hydroxylauric acid); 2-hydroxytetradecanoic acid (alpha hydroxymyristic acid); 2-hydroxyhexadecanoic acid (alpha hydroxypalmitic acid); 2-hydroxyoctadecanoic acid (alpha hydroxystearic acid); 2-hydroxyeicosanoic acid (alpha hydroxyarachidonic acid); 2-phenyl 2-hydroxyethanoic acid (mandelic acid); 2,2-diphenyl 2-hydroxyethanoic acid (benzilic acid); 3-phenyl 2-hydroxypropanoic acid (phenyl)acetic acid); 2-phenyl 2-methyl 2-hydroxyethanoic acid (atrolactic acid, 2-(4′-hydroxyphenyl); 2-hydroxyethanoic acid (4-hydroxymandelic acid); 2-(4′-chlorophenyl) 2-hydroxyethanoic acid (4-chloromandelic acid); 2-(3′-hydroxy-4′-methoxyphenyl) 2-hydroxyethanoic acid (3-hydroxy-4-methoxymandelic acid); 2-(4′-hydroxy-3′-methoxyphenyl); 2-hydroxyethanoic acid (4-hydroxy-3-methoxymandelic acid); 3-(2′-hydroxyphenyl); 2-hydroxypropanoic acid (3-(2′-hydroxy phenyl) lactic acid); 3-(4′-hydroxyphenyl) 2-hydroxypropanoic acid (3-(4′-hydroxyphenyl) lactic acid)); 2-(3′,4′-dihydroxyphenyl) 2-hydroxyethanoic acid (3,4-dihydroxymandelic acid).

Polyhydroxy AHAs suitable for use in the present invention include: 2,3-dihydroxypropanoic acid (glyceric acid); 2,3,4-trihydroxybutanoic acid and its isomers (erythronic acid, threonic acid); 2,3,4,5-tetrahydroxypentanoic acid and its isomers (ribonic acid, arabinoic acid, xylonic acid, lyxonic acid); 2,3,4,5,6-pentahydroxyhexanoic acid and its isomers (allonic acid, altronic acid, gluconic acid, mannoic acid, gulonic acid, idonic acid, galactonic acid, talonic acid); 2,3,4,5,6,7-hexahydroxyheptanoic acid and its isomers (glucoheptonic acid, galactoheptonic acid).

Polycarboxylic AHAs suitable for use in the present invention include: 2-hydroxypropane-1,3-dioic acid (tartronic acid); 2-hydroxybutane-1,4-dioic acid (malic acid); 2,3-dihydroxybutane-1,4-dioc acid (tartaric acid); 2-hydroxy-2-carboxypentane-1,5-dioic acid (citric acid); 2,3,4,5-tetrahydroxyhexane-1,6-dioic acid and its isomers (saccharic acid, mucic acid).

AHAs may be used in the combination therapies of the present invention at concentrations ranging from about 0.1% to about 10%, more preferably from about 0.2% to about 5%, also preferably from about 0.5% to about 2%.

Combination therapies according to the present invention may comprise topical administration of compositions containing a safe and effective amount of salicylic acid, its esters, its salts, or combinations thereof. In compositions according to this aspect of the present invention, the salicylic acid compound (including esters, salts and mixtures) preferably comprises from about 0.0001% to about 25%, more preferably from about 0.001% to about 15%, even more preferably from about 0.01% to about 10%, still more preferably from about 0.1% to about 5%, and even more preferably from about 0.2% to about 2%, by weight of the composition, of the salicylic acid compound.

In one embodiment of this first aspect of the present invention, the hydroxyacid may be included in the same formulation as the imidazoquinoline amine derivative of Formula 1.

In another embodiment of the first aspect of the present invention, in addition to the administration of safe and effective amounts of (i) an imidazoquinoline amine derivative according to Formula 1 and (ii) one or more of a retinoid and/or hydroxyacid, a patient with fine lines or clinical wrinkles on a section of aged skin or non-precancerous, normal photodamaged skin is also treated with (iii) one or more dermatocosmetic active ingredients selected from the group consisting of ingredients that (a) help to reduce the appearance of and/or prevent the formation of fine lines and/or wrinkles; and/or (b) reduce transepidermal water loss; and/or (c) improve skin retention of moisture; and/or (d) improve skin elasticity. Non-limiting examples of such dermatocosmetic actives include: anti-inflammatory agents; antioxidants; vitamins and derivatives thereof; exfoliants (including abrasive particles); skin soothing agents (e.g., panthenol and its derivatives, aloe vera, pantothenic acid and its derivatives, allantoin, bisabolol, and dipotassium glycyrrhizinate); short-chain peptides, (i.e., having less than about 12 amino acids), including lipopeptides; growth factors; conjugated linoleic acid; skin lightening/bleaching/depigmenting agents, including tyrosinase inhibitors and PAR-2 agonists and/or antagonists; and agents that block or attenuate ultraviolet radiation.

In a preferred embodiment of aspects of the present invention in which retinoids are topically administered, the patient undergoing the combination therapy also applies a topical product containing a combination of the following sunscreens and sunblocks: p-Aminobenzoic acid up to 15%; Avobenzone up to 3%; Cinoxate up to 3%; Dioxybenzone up to 3%; Homosalate up to 15%; Menthyl anthranilate up to 5%; Octocrylene up to 10%; Octylmethoxycinnamate (Octinoxate) up to 7.5%; Octyl salicylate up to 5%; Oxybenzone up to 6%; Padimate 0 up to 8%; Phenylbenzimidazole sulfonic acid (Ensulizole) up to 4%; Sulisobenzone up to 10%; Titanium dioxide up to 25%; Trolamine salicylate up to 12%; Zinc oxide up to 25%. The foregoing sunscreens are currently approved by the U.S. Food and Drug Administration. Other sunscreens and sunblocks approved in countries outside the U.S. are also suitable for use according to this aspect of the invention.

Additional active ingredients suitable for use in topical formulations in the combination therapies of the present invention are described in international patent application publication WO/2007/100689 and U.S. Pat. Nos. 6,492,326 and 6,277,892 and U.S. Patent Application Publication Nos. 2005/0142095 and 2004/0180020, the disclosures of which are each incorporated by reference herein in their entirety.

A second aspect of the present invention is directed to topical treatments according to the first aspect of the invention in combination with cosmetic procedures performed by a licensed medical professional or allied health professional (i.e., an individual who has completed requisite schooling and/or training as mandated by a state regulatory body and is licensed to provide aesthetic/cosmetic treatments).

In one embodiment of this aspect of the present invention, the cosmetic procedure used to treat photodamaged skin is a medium or deep chemical peel performed in the office of a physician, licensed aesthetician, day spa or medispa under the supervision of a physician. The deep chemical peel may consist of application of one or more exfoliating agents selected from the group consisting of glycolic acid, salicylic acid, trichloroacetic acid and phenol, and combinations thereof. In one preferred embodiment, glycolic acid may be used at concentration of from about 30% to about 70%. In another preferred embodiment, trichloroacetic acid (“TCA”) may be used at concentration of from about 10% to about 70%, preferably at a concentration of about 35%. TCA (at a concentration of from about 70% to about 75%) may also be used in combination with glycolic acid (at a concentration of from about 30% to about 35%). Additionally, Jessner's solution (resorcinol, salicylic acid and lactic acid) may be used alone or followed by TCA.

In another embodiment of this aspect of the present invention, the cosmetic procedure used to treat photodamaged skin is mechanical exfoliation, where the procedure is selected from the group consisting of dermabrasion and microdermabrasion.

In yet another embodiment of this aspect of the present invention, the cosmetic procedure involves the administration of light and is selected from the group consisting of: laser skin treatment, where the method of laser skin treatment is selected from the group consisting of ablative laser skin treatments (CO₂ and Erbium:YAG lasers), non-ablative laser skin treatments, and fractional laser skin treatments; Light Emitting Diode (L.E.D.) Light Therapy; and Intense Pulsed Light (I.P.L.) Therapy.

The following are illustrative examples of topical formulations that can be used in the combination therapies of the present invention. The components and specific ingredients are presented as being typical, and various modifications can be derived in view of the foregoing disclosure within the scope of the invention.

### Example 1

### Imiguimod/AHA Cream

| Sequence | INCI NAME | Percent |
|---|---|---|
| 1 | Water | 28.10 |
| 1 | Xanthan Gum | 30.00 |
| 1 | Disodium EDTA | 0.10 |
| 1 | Phenoxyethanol and Methylparaben and | 0.50 |
| | Ethylparaben and Butylparaben and | |
| | Propylparaben (and) Isobutylparaben | |
| 1 | Sodium Dehydroacetate | 0.10 |
| 1 | Glycereth-26 | 2.00 |
| 1 | Magnesium Aluminum Silicate | 15.00 |
| 1 | Triethanolamine | 0.40 |
| 2 | Stearyl Alcohol (and) Ceteareth-20 | 1.00 |
| 2 | Neopentyl Glycol Dicaprylate/Dicaprate | 4.00 |
| 2 | Hydrogenated Polyisobutene | 1.50 |
| 2 | Stearic Acid | 4.00 |
| 2 | Glyceryl Stearate (and) PEG-100 Stearate | 2.50 |
| 2 | Sesamum Indicum (Sesame) Oil | 2.00 |
| 2 | Cetyl Alcohol | 1.50 |
| 2 | Dimethicone | 0.50 |
| 2 | Hydrogenated Vegetable Oil | 0.50 |
| 4 | Glycolic Acid | 2.00 |
| 5 | Butylene Glycol | 3.00 |
| 5 | Imiquimod | 1.25 |
| 5 | Glycyrrhiza Glabra (licorice) Extract | 0.05 |
| 6 | Triethanolamine | Q.S. |

Combine Sequence #1 ingredients and heat to 78-80° C. with propeller mixing. Heat Sequence #2 ingredients to 80° C. and add Sequence #1 with propeller mixing. Cool to 45° C. and add Sequence #3 and Sequence #4 to batch with moderate speed propeller mixing. Combine Sequence #5 ingredients and heat to 40-50° C. Mix until clear and uniform. Add Sequence #5 to batch and mix well. Cool to 25° C. and adjust pH to 3.8-4.2.

### Example 2

### Imiguimod/AHA Cream

| Sequence | INCI Name | Percent |
|---|---|---|
| 1 | Water | 45.38 |
| 1 | Bentonite (and) Xanthan Gum | 2.00 |
| 2 | Glycereth-26 | 2.50 |
| 2 | Phenoxyethanol (and) Methylparaben (and) | 0.75 |
| | Ethylparaben (and) Butylparaben (and) | |
| | Propylparaben (and) Isobutylparaben | |
| 2 | PEG-8 | 3.00 |
| 2 | Butylene Glycol | 5.00 |
| 2 | Triethanolamine | 3.00 |
| 3 | Ceteryl Alcohol (and) Ceteareth-20 | 5.80 |
| 3 | Cetyl Alcohol | 1.40 |
| 3 | Stearic Acid | 0.60 |
| 3 | Glyceryl Stearate SE | 1.00 |
| 3 | Sorbitan Palmitate | 1.75 |
| 3 | Polysorbate-40 | 3.00 |
| 3 | Hydrogenated Polyisobutene | 2.50 |
| 3 | Myristyl Myristate | 1.00 |
| 3 | Tridecyl Trimellitate | 1.00 |
| 3 | Dimethicone | 0.75 |
| 3 | Tocopheryl Acetate | 0.02 |
| 4 | Glycolic Acid | 5.00 |
| 4 | Water | 5.00 |
| 5 | Methylchlorolsothiozolinone (and) | 0.05 |
| | Methylisothiazolinone | |
| 6 | Water | 5.00 |
| 6 | Imiquimod | 2.50 |
| 7 | Butylene Glycol | 1.50 |
| 7 | Menthol | 0.50 |
| 8 | Triethanolamine | QS |

Heat the water (Sequence #1) to 78-80° C. Add Sequence #1 ingredients under propeller mixer and mix until complete dissolution. Premix Sequence #2 ingredients and add them to Sequence #1. Heat Sequence #3 ingredients to 78-80° C., mix and add to the main vessel under homogenizer. Mix for approximately 3-5 minutes and switch to propeller mixer. Cool to 45° C. and add Sequence #4 and Sequence #5 to batch with propeller mixer. Cool to 35-40° C. and add premixed Sequence #6, mix thoroughly, and add premixed Sequence #7 to the batch. Cool batch to 25° C. and adjust pH to 3.8-4.2 using Sequence #8.

### Example 3

### Imiguimod Gel

| Phase | INCI Name | % |
|---|---|---|
| A | Carbomer | 0.5 |
| A | Water | 90.5 |
| B | Imiquimod | 5.0 |
| C | Helianthus Annus (Sunflower) Seed Extract | 1.0 |
| C | Panthenol | 1.0 |
| C | Aloe Vera | 1.0 |
| D | Phenoxyethanol (and) Methylparaben (and) | 1.0 |
| | Ethylparaben (and) Butylparaben (and) | |
| | Propylparaben (and) Isobutylparaben | |

Combine Phase A ingredients and mix until uniform at room temperature. Add Phase B to Phase A with mixing. Add Phase C ingredients to Phase A/B while mixing. Add Phase D to Phase A/B/C and mix well.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

1. A method of reducing the appearance of fine lines and wrinkles on aged skin or non-precancerous, normal photodamaged section of skin, in a patient not being treated for viral infection or skin cancer at the same section of the skin, comprising
(a) topically applying in a dermatologically-acceptable carrier, a safe and effective amount of an imidazoquinoline amine derivative conforming to the structure 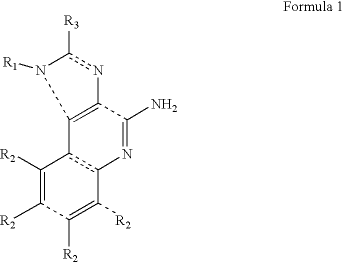
wherein
(i) R₁ is selected from the group consisting of C₁-C₁₀ alkyl; C₁-C₆ hydroxylalkyl; and acyloxyalkyl wherein the acyloxy moiety is C₂-C₄ alkanoyloxy or benzoyloxy, and the alkyl moiety contains one to six carbon atoms or a benzyl, (phenyl)ethyl or phenyl
(ii) R₂ is hydrogen or no more than two non-hydrogen moieties selected from the group consisting of C₁-C₄ alkyl, C₁-C₄ alkoxy, and halogen with the proviso that non-hydrogen moieties are present then said moieties together contain no more than 6 carbon atoms;
(iii) R₃ is selected from the group consisting of hydrogen, C₁-C₈ alkyl, benzyl, (phenyl)ethyl and phenyl; and
(iv) benzyl, (phenyl)ethyl and phenyl; and
(b) topically applying, in a dermatologically-acceptable carrier, a safe and effective amount of a retinoid and/or hydroxyacid; and/or
(c) administering to a section of aged skin or non-precancerous, normal photodamaged skin having fine lines or clinical wrinkles one or more cosmetic treatments selected from the group consisting of:
(i) Light Emitting Diode (L.E.D.) Light Therapy;
(ii) Intense Pulsed Light (I.P.L.) Therapy;
(iii) laser skin resurfacing;
(iv) mechanical exfoliation;
(v) superficial, medium depth or deep chemical peels;
(vi) radiofrequency treatment;
(vii) ultrasound treatment;
(viii) intradermal and intraepidermal injections with hyaluronic acid and derivatives thereof; and
(ix) cryosurgery.

2. A method of claim 1 wherein the imidazoquinoline amine derivative according to Formula 1 is 1-isobutyl-1H-imidazo[4,5-c]quinolin-4-amine.

3. A method of claim 2 wherein 1-isobutyl-1H-imidazo[4,5-c]quinolin-4-amine is administered at a concentration of from about 0.1% to about 5.0%.

4. A method of claim 3 wherein the hydroxyacid is an alpha-hydroxy acid.

5. A method of claim 4 wherein the alpha-hydroxy acid is selected from the group consisting of lactic acid, glycolic acid and salicylic acid and mixtures thereof.

6. A method of claim 3 wherein the retinoid is selected from the group consisting of retinol, retinyl palmitate, retinyl acetate, retinyl propionate, retinal and combinations thereof.

7. A method of claim 3 wherein 1-isobutyl-1H-imidazo[4,5-c]quinolin-4-amine, a retinoid and a hydroxy acid are each administered at safe and effective concentrations.

8. A method of claim 3 further comprising the topical administration of a safe and effective amount an antioxidant, a growth factor or a sunscreen or sunblock.
